(19) United States
(12) Patent Application Publication
(10) Pub. No.: US 2019/0360918 A1
(43) Pub. Date: Nov. 28, 2019

(54) Method for Analyzing Acrylic Acid Content in Acrylic Adhesive Resin Copolymer

(71) Applicant: LG Chem, Ltd., Seoul (KR)

(72) Inventors: Dong Hyun Kim, Daejeon (KR); Byoung Hyoun Kim, Daejeon (KR); Su Youn Han, Daejeon (KR)

(73) Assignee: LG Chem, Ltd., Seoul (KR)

(21) Appl. No.: 16/477,679

(22) PCT Filed: Feb. 1, 2018
(86) PCT No.: PCT/KR2018/001373

§ 371 Date: Jul. 12, 2019

(30) Foreign Application Priority Data
Jul. 28, 2017 (KR) 10-2017-0096308

(51) Int. Cl. G01N 19/10; C09J 133/08; C08K 5/23; G01N 33/44; C08F 2/06; C08F 220/06; C08F 220/18; C09J 133/02

(52) U.S. Cl. CPC G01N 19/10; C09J 133/08; C08K 5/235; G01N 33/442; C08F 2220/1841; C08F 220/06; C08F 220/18; C09J 133/02; C08F 2220/1825; C08F 2/06

(57) ABSTRACT

A method enabling the quantitative analysis of acrylic acid in an acrylic adhesive resin includes: measuring, by means of a moisture analyzer for solid samples (MASS), moisture content generated through a ring forming reaction, in a high-temperature environment, of an acrylic polymer having a carboxyl group; and analyzing the acrylic acid content on the basis of the measured moisture content and the amount of the sample that is used.

### CROSS-REFERENCE TO RELATED APPLICATIONS

This application is a national stage entry under 35 U.S.C. § 371 of International Application No. PCT/KR2018/001373, filed Feb. 1, 2018, which claims priority to Korean Patent Application No. 10-2017-0096308, filed Jul. 28, 2017, the disclosures of which are incorporated herein by reference.

### TECHNICAL FIELD

The present invention relates to an analysis method of acrylic acid content in an acrylic adhesive resin copolymer.

### BACKGROUND ART

It is known that the amount of acrylic acid in an acrylic adhesive resin copolymer is a main factor affecting the properties of adhesives (see Polymer Testing 27 (2008) 870: International Journal of Adhesion & Adhesives 34 (2012) 107). In order to analyze such an acrylic acid, the presence of acrylic acid have been confirmed by using FT-IR (Fourier transform infrared) or the content of acrylic acid have been analyzed by using GC (gas chromatography) (see Polymer Testing 27 (2008) 870). However, there were examples that the FT-IR exhibited the presence of acrylic acid even in the case that the acrylic acid is not present. Also, there is a trial of using an EGA-GC/MS (Evolved Gas Analysis-Gas Chromatography/Mass Spectrometry) wherein an acrylic acid content is confirmed by measuring the amount of moisture generated from the acrylic polymer having carboxylic groups by a ring formation reaction under a high temperature environment. However, this is not proper in the analysis of acrylic acid content due to problems such as water obstruction and peak overlapping detected in the EGA-GC/MS itself.

Meanwhile, in the case of using an MASS (Moisture Analyzer for Solid Sample) for analyzing an acrylic acid content, it requires removal of moisture from outside, and optimization of a temperature condition that makes the completion of the ring formation reaction of carboxylic groups generating moisture and the amount of a sample that allows good heat transfer in the sample.

The present inventors have endeavored to solve the above problems and found that the amount of moisture generated from the acrylic polymer having carboxylic groups by a ring formation reaction under a high temperature environment is measured by using a moisture analyzer for solid sample (MASS), and then the acrylic acid content can be analyzed based on the measured amount of moisture and the used amount of the sample.

### DETAILED DESCRIPTION OF THE INVENTION

### Technical Problem

It is an aspect of the present invention to provide an analysis method of an acrylic acid content, which uses to generate the ring formation reaction of an acrylic polymer having carboxylic groups under a high temperature environment.

### Technical Solution

An acrylic polymer having carboxylic groups generates moisture during the ring formation reaction of the carboxylic groups under a high temperature environment as follows:

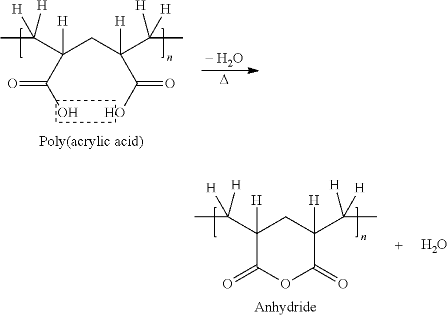

The present invention measures the amount of moisture generated during the ring formation reaction of the carboxylic groups using an MASS which is self-manufactured and calculates an acrylic acid content from the measurement of the moisture amount, and the calculated acrylic acid content is confirmed to correspond with the results of ¹H HR MAS NMR experiments.

In order to accomplish the technical solution, the present invention provides analysis method of an acrylic acid content by measuring the amount of moisture generated from an acrylic adhesive resin copolymer using an MASS and calculates an acrylic acid content based on the measured amount of moisture and the amount of the sample used.

In one embodiment, the present invention provides a method for determining a content of an acrylic acid in an acrylic adhesive resin copolymer, comprising: measuring the amount of moisture generated from the acrylic adhesive resin copolymer obtained by polymerization of two or more acrylic acid-based monomers using a moisture analyzer for solid sample (MASS), and calculating the content of the acrylic acid by inserting the measured amount of moisture into the following Equation 1:

$$AA Content ( \% ) = \frac{{W}_{H 2 O} \times 2 \times {MW}_{AA}}{1000 \times {MW}_{H 2 O} \times {W}_{S}} \times 100 \qquad [ Equation 1 ]$$

wherein,

AA represents the acrylic acid,

W_{H2O} is a measured amount (μg) of moisture,

“2” in a numerator represents that 1 mole of H₂O is generated per 2 moles of acrylic acids,

MW_{AA} is a molecular weight (72.06 g/mol) of acrylic acid,

“1000” in a denominator is for calibration of a weight unit,

MW_{H2O} is a molecular weight (18.02 g/mol) of H₂O, and

W_{S} is an amount (mg) of a sample of the acrylic copolymer adhesive resin

In one embodiment, the amount of moisture is measured by using the MASS at a final temperature ranging from 355 to 365° C. If the final temperature of the MASS measurement is 350° C., the ring formation reaction is insufficiently carried out and the amount of moisture is measured to be low. If the final temperature is 370° C., moisture is further generated by a ring cleavage reaction after ring formation and the moisture value is evaluated more than the actual amount.

In one embodiment, the two or more acrylic acid-based monomers comprise acrylic acid (AA), ethyl hexyl acrylate (EHA), butyl acrylate (BA), methyl acrylate (MA), ethyl acrylate (EA), ethyl hexyl methacrylate (EHMA), butyl methacrylate (BMA), methyl methacrylate (MMA) or ethyl methacrylate (EMA).

In one embodiment, the two or more acrylic acid-based monomers comprise acrylic acid (AA), ethyl hexyl acrylate (EHA) or butyl acrylate (BA).

In one embodiment, the acrylic adhesive resin copolymer is made in the form of a film by polymerization of the monomers, a solvent and a thermal radical initiator (TRI).

In one embodiment, the solvent comprises ethyl acetate (EtOAc) or solvents having a boiling point of 60 to 78° C.

In one embodiment, the TRI comprises azobis(isobutyronitrile) (AIBN) or diazo compounds.

In one embodiment, the polymerization is carried out at a temperature of 60 to 78° C.

### Advantageous Effects

The quantitative analysis method of an acrylic acid content according to the present invention can provide a reproducible analysis value of acrylic acid content corresponding with the results of NMR experiments, while avoiding demerits of the conventional methods.

### BEST MODE

Hereinafter, the present invention will be described in detail.

It should be understood that the terms used in the specification and the appended claims should not be construed as limited to general and dictionary meanings, but interpreted based on the meanings and concepts corresponding to technical aspects of the present invention on the basis of the principle that the inventor is allowed to define terms appropriately for the best explanation.

The method for determining a content of an acrylic acid according to the present invention comprises measuring the amount of moisture generated from the acrylic adhesive resin copolymer obtained by polymerization of two or more acrylic acid-based monomers by using a moisture analyzer for solid sample

(MASS), and calculating the content of the acrylic acid by inserting the measured amount of moisture into the following Equation 1:

$$AA Content ( \% ) = \frac{{W}_{H 2 O} \times 2 \times {MW}_{AA}}{1000 \times {MW}_{H 2 O} \times {W}_{S}} \times 100 \qquad [ Equation 1 ]$$

wherein,

AA represents the acrylic acid,

W_{H2O} is a measured amount (μg) of moisture,

“2” in a numerator represents that 1 mole of H₂O is generated per 2 moles of acrylic acids,

MW_{AA} is a molecular weight (72.06 g/mol) of acrylic acid,

“1000” in a denominator is for calibration of a weight unit,

MW_{H2O} is a molecular weight (18.02 g/mol) of H₂O, and

W_{S} is an amount (mg) of a sample of the acrylic copolymer adhesive resin.

In one embodiment, the amount of moisture is measured by using the MASS at a final temperature ranging from 355 to 365° C. If the final temperature of the MASS measurement is 350° C., the ring formation reaction is insufficiently carried out and the amount of moisture is measured to be low. If the final temperature is 370° C., moisture is further generated by a ring cleavage reaction after ring formation and the moisture value is evaluated more than the actual amount.

In one embodiment, the two or more acrylic acid-based monomers comprise acrylic acid (AA), ethyl hexyl acrylate (EHA), butyl acrylate (BA), methyl acrylate (MA), ethyl acrylate (EA), ethyl hexyl methacrylate (EHMA), butyl methacrylate (BMA), methyl methacrylate (MMA) and ethyl methacrylate (EMA), for example two or more acrylic acids selected from the group consisting of acrylic acid (AA), ethyl hexyl acrylate (EHA) or butyl acrylate (BA).

In one embodiment, the acrylic adhesive resin copolymer is made in the form of a film by polymerization of the monomers, a solvent and a thermal radical initiator (TRI).

In one embodiment, the solvent comprises ethyl acetate (EtOAc) or solvents having a boiling point of 60 to 78° C.

In one embodiment, the TRI comprises azobis(isobutyronitrile) (AIBN) or diazo compounds.

In one embodiment, the polymerization is carried out at a temperature of 60 to 78° C.

The acrylic acid content determined by the quantitative analysis method according to the present invention is well matched with the used amount of each monomer and is substantially corresponded with the results of ¹H HR MAS NMR experiments.

Hereinafter, the present invention will be described in more detail with reference to Examples. It will be apparent to those skilled in the art that the following examples are intended to be illustrative of the present invention and not to be construed as limiting the scope of the invention.

### Example

Preparation of Acrylic Adhesive Resin Copolymer Sample

As listed in Table 1, 6 acrylic adhesive resin samples in which the type and content ratio of monomers (wt/wt) were each different, i.e., B99A1 (BA 99%/AA 1%), B95A5 (BA 95%/AA 5%), B90A10 (BA 90%/AA 10%), E99A1 (EHA 99%/AA 1%), E95A5 (EHA 95%/AA 5%) and E90A10 (EHA 90%/AA 10%), were prepared by adding the corresponding monomers in EtOAc as a solvent for 1 hour, to which AIBN is added as a TRI, followed by polymerization. The polymerization was carried out 78° C., the boiling point of EtOAc for 6 hours to give a copolymer conversion of 99.7%. After polymerization, the resulting copolymer was added with a solvent of EtOAc to control its viscosity (1000 to 2000 cP) which is suitable for coating, thereby obtaining a coating solution for forming a film. After coating, drying was carried to remove the solvent. The coating solution generally had a total solid content (TSC) of 10% to 20%.

In the coating procedure, the copolymer solution was coated on the surface of a release film in a thickness of 25 μm by using a bar coater and dried in an oven (convection oven) set to 120 t for 3 minutes to remove the solvent EtOAc and the unreacted monomer. After drying, another release film having different peel strength was laminated on the coated surface to form a film having the release films on both sides.

TABLE 1
Monomer Composition of 6
acrylic adhesive resin samples

| Acrylic | wt % | | |
|---|---|---|---|
| Adhesive | ¹⁾BA | ²⁾EHA | ³⁾AA |
| B99A1 | 99 | 0 | 1 |
| B95A5 | 95 | 0 | 5 |
| B90A10 | 90 | 0 | 10 |
| E99A1 | 0 | 99 | 1 |
| E95A5 | 0 | 95 | 5 |
| E90A10 | 0 | 90 | 10 |

¹⁾BA: Butyl Acrylate
²⁾EHA: 2-Ethyl Hexyl Acrylate
³⁾AA: Acrylic Acid

Analysis Method and Conditions

(1) EGA-MS (Evolved Gas Analysis-Mass Spectrometry) Analysis

EGA-MS analysis was carried out to confirm the temperature range in which moisture was generated from the acrylic adhesive. The apparatus used for the EGA-MS analysis was a double-shot pyrolyzer (PY-2020id) manufactured by Frontier Laboratories and GC/MSD (Agilent 7890A GC system/5975C inert XL mass selective detector) equipped with ALLOY-DTM (deactivated metal column) (0.15 mm (I.D.)×2.5 m (L), coated film thickness <0.01 μm). The pyrolyzer was maintained at 50° C. for 5 minutes and its temperature was raised to 600° C. by the rise rate of 10° C. per minute to give a program. The flow rate of a carrier gas (He) was 1.0 mL/min and the split ratio was 20:1. The temperature of an injector, an oven and an interface in the GC/MSD was 300° C., the temperature of the pyrolyzer interface was 320° C., and the scan range was from 15 to 700 amu.

(2)¹H HR MAS NMR (High Resolution Magic Angle Spinning Nuclear Magnetic Resonance) Analysis

This analysis was performed using the apparatus of Agilent 600 MHz SSNMR (Solid State Nuclear Magnetic Resonance) equipped with a NANO probe at room temperature (25° C.). ¹H HR MAS experiments were carried out by using an s2pul pulse sequence under the conditions of scan number=16, relaxation delay=5 seconds, pulse width=9.5 μsec, acquisition time=1.7 seconds, and spinning rate=2.3 kHz. The samples were loaded on CDCl₃ in a ¹H HR MAS rotor and swelled for 3 hrs or more before measurement.

(3) Karl Fischer (K/F) Titrator

The K/F titrator was used with HYDRANAL-Coulomat AG-Oven (Sigma-Aldrich, Cat. No. 34739-500ML-R) as a reagent. The extraction time of the K/F titrator was set to 300 seconds, the start drift value was set to 20 μg/min, the stop time was set to OFF, and the initial drift value was 10 μg/min or less.

(4) Measurement of Moisture Amount in Standard Material Using MASS (Moisture Analyzer for Solid Sample)

1% K/F oven standard (Na₂WO₄.2H₂O) was used as a standard material and the flow rate of helium as set to 100 mL/min. The temperature condition had two intervals. The first temperature interval was maintained at 50° C. for 5 minutes, and the valve was set to the OFF state to purge the vaporized substances. The second temperature interval was set to raise from 50° C. to 450° C. for 35 minutes, and the valve was set to the ON state so that the vaporized substances were introduced into the K/F titrator.

(5) Measurement of Moisture Amount in Sample Using MASS (Moisture Analyzer for Solid Sample)

Similar to the measurement of moisture amount in standard material, the flow rate of helium was set to 100 mL/min. The temperature condition had four intervals. The first and second temperature intervals were set to rise from room temperature to 130° C. for 5 minutes and maintained at 130° C. for 5 minutes, and the valve was set to the OFF state in the two intervals to purge the vaporized substances. The third and fourth temperature intervals were set to rise from 130° C. to 360° C. for 5 minutes and maintained at 360° C. for 10 minutes, and the valve was set to the ON state in the two intervals so that the vaporized substances were introduced into the K/F titrator.

The aliquots of the samples in the amount of 15 to 25 mg were exactly weighed in the unit of 0.1 mg and applied to the experiments. The content of an acrylic acid in the samples was calculated by using the content (μg) of moisture measured via the K/F titrator.

Calculation of Acrylic Acid Content

The acrylic acid content in the acrylic adhesive resin samples was calculated by measuring the amounts of the acrylic adhesive resin samples and the amount of moisture generated therefrom by using the MASS, and inserting the measurements into the following Equation 1:

$$AA Content ( \% ) = \frac{{W}_{H 2 O} \times 2 \times {MW}_{AA}}{1000 \times {MW}_{H 2 O} \times {W}_{S}} \times 100 \qquad [ Equation 1 ]$$

wherein,

AA represents the acrylic acid,

W_{H2O} is a measured amount (μg) of moisture,

“2” in a numerator represents that 1 mole of H₂O is generated per 2 moles of acrylic acids,

MW_{AA} is a molecular weight (72.06 g/mol) of acrylic acid,

“1000” in a denominator is for calibration of a weight unit,

MW_{H2O} is a molecular weight (18.02 g/mol) of H₂O, and

W_{S} is an amount (mg) of a sample of the acrylic copolymer adhesive resin.

The amount of moisture was measured by using the MASS at a final temperature ranging from 355 to 365° C.

Analysis Results

(1) MASS Analysis

The results of acrylic acid content in the acrylic adhesive resin samples measured by using the MASS are shown in Table 2 below.

From the RSD values of Table 2 below, it was confirmed that all of 6 acrylic adhesive resin samples exhibited reproducible results. In the acrylic acid content (“Average”), BA-based samples (B99A1, B95A5 and B90A10) were well matched with the feed of the monomers used for their preparation, while EHA-based samples (E99A1, E95A5 and E90A10) exhibited a difference of 0.3% to 0.5% as compared with the feed of the monomers.

TABLE 2
Analysis Results of Acrylic
Acid Content by Mass

| | Feed | Results | Average | RSD |
|---|---|---|---|---|
| Sample | (AA, wt %) | (AA, wt %) | (n = 3) | (%) |
| B99A1 | 1.0 | 0.9 | 1.0 | 15.9 |
| | | 1.0 | | |
| | | 1.2 | | |
| B95A5 | 5.0 | 5.0 | 5.0 | 1.2 |
| | | 4.9 | | |
| | | 5.0 | | |
| B90A10 | 10.0 | 10.3 | 10.0 | 2.8 |
| | | 10.1 | | |
| | | 9.7 | | |
| E99A1 | 1.0 | 1.3 | 1.3 | 0.0 |
| | | 1.3 | | |
| | | 1.3 | | |
| E95A5 | 5.0 | 4.7 | 4.5 | 4.0 |
| | | 4.3 | | |
| | | 4.6 | | |
| E90A10 | 10.0 | 10.3 | 10.4 | 0.5 |
| | | 10.4 | | |
| | | 10.4 | | |

(2)¹H HR MAS NMR Analysis

¹H HR MAS NMR analysis was performed in order to confirm the accuracy of the acrylic acid content values in the acrylic adhesive resin samples as shown in Table 2 above, which were obtained from the MASS experiment. The results thereof are shown in Table 3 below.

Comparing the MASS results shown in Table 2 above with the ¹H HR MAS NMR results shown in Table 3 below regarding the acrylic acid content, the samples excluding E95A5 (EHA 95%/AA 5%) exhibited a difference within 0.5%, i.e., 0.1%, 0.2% or 0.4%, while E95A5 (EHA 95%/AA 5%) exhibited a difference of 1.0%, specifically 4.5 wt % (MASS) and 5.5 wt % (NMR). It is understood that there was an error due to moisture peaks detected during the ¹H HR MAS NMR experiment.

TABLE 3
Analysis Results of Acrylic Acid
Content by ¹H HR MAS NMR

| | Feed | Content ratio (wt/wt) by | | |
|---|---|---|---|---|
| | (AA, | ¹H HR MAS NMR | | |
| Sample | wt %) | AA | BA | EHA |
| B99A1 | 1.0 | 1.1 | 98.9 | — |
| B95A5 | 5.0 | 5.2 | 94.8 | — |
| B90A10 | 10.0 | 10.4 | 89.6 | — |
| E99A1 | 1.0 | 1.2 | — | 98.8 |
| E95A5 | 5.0 | 5.5 | — | 94.5 |
| E90A10 | 10.0 | 10.9 | — | 89.1 |

From the MASS results shown in Table 2 above with the ¹H HR MAS NMR results shown in Table 3 above regarding the acrylic acid content, it was confirmed that the present invention can provide reproducible analysis values of acrylic acid content by measuring the amount of moisture generated from the acrylic adhesive resin copolymer by using the MASS, followed by calculation inserting the measured amount of moisture and the used amount of the acrylic adhesive resin copolymer into Equation 1, and the obtained content was corresponded with the results of NMR experiments.

While the present invention has been particularly shown and described with reference to embodiments thereof, it will be understood by those of ordinary skill in the art that the scope of the present invention is not limited thereby and that various changes and modifications may be made therein. Therefore, the actual scope of the present invention will be defined by the appended claims and their equivalents.

1. A method for determining a content of an acrylic acid in an acrylic adhesive resin copolymer, comprising:
measuring an amount of moisture generated from the acrylic adhesive resin copolymer obtained by polymerization of two or more acrylic acid-based monomers by using a moisture analyzer for solid sample (MASS) to obtain a measured amount of moisture, and
calculating the content of the acrylic acid by inserting the measured amount of moisture into the following Equation 1:
$$AA Content ( \% ) = \frac{{W}_{H 2 O} \times 2 \times {MW}_{AA}}{1000 \times {MW}_{H 2 O} \times {W}_{S}} \times 100 \qquad [ Equation 1 ]$$
wherein,
AA represents the acrylic acid,
W_{H2O} is the measured amount (m) of moisture,
“2” in a numerator represents that 1 mole of H₂O is generated per 2 moles of acrylic acids,
MW_{AA} is a molecular weight (72.06 g/mol) of acrylic acid,
“1000” in a denominator is for calibration of a weight unit,
MW_{H2O} is a molecular weight (18.02 g/mol) of H₂O, and
W_{S} is an amount (mg) of a sample of the acrylic adhesive resin copolymer.

2. The method of claim 1, wherein a final temperature at which the amount of moisture is measured by using the MASS is from 355 to 365° C.

3. The method of claim 1, wherein the two or more acrylic acid-based monomers comprise acrylic acid (AA), ethyl hexyl acrylate (EHA), butyl acrylate (BA), methyl acrylate (MA), ethyl acrylate (EA), ethyl hexyl methacrylate (EHMA), butyl methacrylate (BMA), methyl methacrylate (MMA) or ethyl methacrylate (EMA).

4. The method of claim 3, wherein the two or more acrylic acid-based monomers comprise acrylic acid (AA), ethyl hexyl acrylate (EHA) or butyl acrylate (BA).

5. The method of claim 1, wherein the acrylic adhesive resin copolymer is made in the form of a film by polymerization of the monomers, a solvent and a thermal radical initiator (TRI).

6. The method of claim 5, wherein the solvent comprises ethyl acetate (EtOAc) or solvents having a boiling point of 60 to 78° C.

7. The method of claim 5, wherein the TRI comprises azobis(isobutyronitrile) (AIBN) or diazo compounds.

8. The method of claim 5, wherein the polymerization is carried out at the boiling point of the solvent.

9. The method of claim 8, wherein the polymerization is carried out at a temperature of 60 to 78° C.
